(19) United States
(12) Patent Application Publication
(10) Pub. No.: US 2023/0255880 A1
(43) Pub. Date: Aug. 17, 2023

(54) IMMOBILIZED ENZYME-BASED WOUND DEBRIDEMENT

(71) Applicant: Guild Associates Inc., Dublin, OH (US)

(72) Inventors: Andrew DINOVO, Columbus, OH (US); Matthew SMIECHOWSKI, Westerville, OH (US); Francis VERHOFF, Cincinnati, OH (US); Dominic DINOVO, Delaware, OH (US)

(21) Appl. No.: 18/170,282

(22) Filed: Feb. 16, 2023

Related U.S. Application Data
(60) Provisional application No. 63/310,752, filed on Feb. 16, 2022.

(51) Int. Cl. A61K 9/00; A61K 45/06; A61P 43/00; A61P 17/02; A61P 31/02; A61P 29/00; A61K 9/06

(52) U.S. Cl. CPC A61K 9/0014; A61K 45/06; A61P 43/00; A61P 17/02; A61P 31/02; A61P 29/00; A61K 9/06

(57) ABSTRACT

The current disclosure provides compositions and methods for treating and/or debriding damaged or dying tissue resulting from a range of injuries and conditions without requiring surgical personnel or facilities. The compositions can be utilized in the field close to where the injury occurred. The compositions include a covalently bonded and immobilized protease enzyme included in a medium that can have a variety of forms and further include a range of additives to further effect the safe and effective removal of dead or damaged tissue. The compositions can be transported and utilized in the field without refrigeration and without any special handling.

### CROSS-REFERENCES TO RELATED APPLICATION

This application claims the benefit of U.S. Provisional Patent Application No. 63/310,752, filed Feb. 16, 2022, and entitled Immobilized Enzyme-Based Wound Debridement which is incorporated herein by reference.

### GOVERNMENT LICENSE RIGHTS

This invention was made with government support under contract W81XWH2010329 awarded by the U.S. Department of Defense.

### BACKGROUND

Following a burn injury, removal of damaged tissue is a critical step in wound healing. The process known as debridement involves the manual removal of infected or necrotic tissue, often through surgery, which allows the skin to better heal and repair as well as prevent life-threatening systemic infection. Timing of debridement is important; delays in this step of burn wound care can lead to poor burn recovery outcomes due to increased risk of infection and slower wound healing from the presence of necrotic tissue. While surgical debridement works well in a hospital setting, those serving in the military and firefighters who suffer burn injuries may have delayed access to a hospital and require immediate burn care and treatment in the field by nonsurgical medical personnel. Non-surgical debridement methods use certain enzymes to break down infected or necrotic tissue and can be effectively utilized by non-medical personnel in a prolonged field care environment.

One of the primary advantages of enzymatic debridement is that it can be applied to burn wounds by non-surgical practitioners. This allows medics or other field personnel to provide early care without needing a surgical theater. By starting treatment quickly, the time an individual is in a hypermetabolic state can be shortened, the likelihood for sepsis and mortality reduced, and overall patient outcomes improved compared to current practices. This timing is significant for individuals injured with limited transport options. Similarly, the enhancements from such a technology improves treatment options for burn wounds that occur during natural disasters, terrorist attacks, or for treating collateral damage to civilian populations in wartime, where the supporting treatment infrastructure is likely overwhelmed or disabled. The invention could also be useful in rural and remote setting where surgical care is uncommon.

Currently only one collagenase-based enzymatic debridement product, Santyl (Smith and Nephew), is FDA approved in the US market. A second product, Nexobrid (Mediwound) using bromelain for debridement, is currently approved in other countries and has recently been approved in the US. Internationally, other enzymes such as papain are also used for debridement. These treatments have been evaluated for their capability to debride wounds and through that action improve wound healing. While these materials work in a hospital or burn center, they are not well suited for military field applications, remote or rural facilities, or for disaster relief as both Santyl and Nexobrid have temperature sensitivities. Santyl must be stored at room temperature but cannot be maintained at temperatures higher than 25° C., cannot be refrigerated, and cannot be stored at cold ambient temperatures. Nexobrid must be stored and transported under refrigeration until the time of use.

Eliminating the temperature sensitivities of current enzyme-based products will enable these treatment options to be carried by combat medics or civilian paramedics so wound debridement can begin at or close to the point of injury and before transport to a care center is available. Enzymatic debridement can also be carried out at a lower cost than surgical debridement in an established health care facility. These features can be combined with advantages that come from enzymatic debridement such as no longer needing to support a surgical theater for effective treatment, opening opportunities for treatment to out-patient, urgent care, and general practice facilities.

A potential hindrance to enzymes in wound healing processes is the presence of matrix metalloproteinases (MMPs) in the wound environment. MMPs are calcium-dependent zinc-containing endopeptidases essential for normal wound healing and other natural processes. The presence of MMPs in the extracellular matrix (ECM) potentially shortens the active lifetime of soluble enzymes whereas immobilized enzymes are protected from protease activity. More recently it has been shown that high levels of MMPs in a wound can result in delayed wound closure, excessive proteolysis, prolonged inflammation, and re-epithelialization failure. The addition of proteolytic-resistant enzymes to a wound, to enhance the degradation of MMPs along with providing a more controlled debridement process would improve the health outcome of a large variety of wounds.

Enzymatic treatments would also benefit if the treatment further exhibited antimicrobial properties to minimize the risk of infection. In the description below such a treatment is described based on the combination of selected immobilized enzymes and their supports formulated in lotions or creams and/or incorporated into a wound pad or dressing.

### SUMMARY

The present disclosure is directed to a composition for improved wound treatment and/or the debridement of damaged, dead, or dying tissue from a wound, injury or medical condition and methods for its use to debride damaged human or animal skin. The composition includes an immobilized protease enzyme further included in a medium. Suitable mediums include, but are not limited to a lotion, a salve, a paste, a cream, a slurry, a pad, a wrap around, and an aerosol. Specific embodiments of the immobilized protease enzyme disclosed are capable of maintaining at least about 50% of its initial activity after storage for at least about 4 months at temperatures ranging from about −20° C. to about +50° C.

Some embodiments can include a further enzyme, either a free enzyme or another immobilized enzyme. For some embodiments involving more than one immobilized enzyme, the individual enzymes can be immobilized on different supports, whereas for other embodiments the individual enzymes can be immobilized on the same support as the initial immobilized protease.

Embodiments of the compositions can also include additional components including, but not limited to an antimicrobial, an analgesic, an anesthetic, or an anti-inflammatory agent. Suitable antimicrobials include, but are not limited to, an antibiotic, an antifungal, an antiviral and an antiseptic. Other embodiments of the compositions can also include a topical anesthetic/analgesic including, but not limited to, lidocaine, bupivacaine, prilocaine, diclofenac, ketoprofen, capsaicin, and aloe vera. Finally, suitable anti-inflammatories include, but are not limited to NSAID's, omega-3 fatty acid, extract of white willow bark, curcumin, ginger, extract of green tea, pycnogenol, Boswellia serrata resin, resveratrol, Uncaria tomentosa, and capsaicin.

Finally, the composition for the wound treatment and/or debridement can also include a medium further including an immobilized enzyme derived from a from a protease enzyme, a crosslinker, and at least one polyfunctional material. The medium can be selected from the group consisting of a lotion, a salve, a paste, a slurry, a cream, an aerosol, a pad and a wrap.

Certain embodiments of the current disclosure include immobilized enzymes that maintain at least 50% of their initial activity for at least 4 months at temperatures ranging from about −20° C. to about +50° C. Other embodiments maintain at least 50% of their initial activity for at least 8 months at temperatures ranging from about −20° C. to about +50° C. Still other embodiments maintain at least 50% of their initial activity for at least 12 months at temperatures ranging from about −20° C. to about +50° C.

Further aspects of the current disclosure include a method for treating and/or debriding damaged tissue from a wound, condition, or disease. Different embodiments of the current disclosure involve applying a medium including an immobilized enzyme to a damaged tissue for treatment and/or debridement purposes where the immobilized enzyme:
is a protease enzyme,
before or after applying, the immobilized enzyme substantially resists degradation,
maintains at least 50% of its initial activity after storage for at least 4 months at temperatures from −20° C. to +50° C.,
deactivates MMPs in the wound, and
debrides damaged tissue contacted.
   In one aspect, the method is directed to the debridement of tissue resulting from a burn where the damage is caused by fire, an explosion, an electrical burn, a chemical burn, and/or a radiation burn. In another aspect, the method is directed to the debridement of damaged tissue resulting from a condition including a chronic wound, a pressure sore, gangrene, elevated MMP concentrations, a diabetic sore, a diabetic ulcer, pressure ulcer, a varicose ulcer, infection, a postoperative wound, a burn, a carbuncle, and a pilonidal cyst wound.

The method can utilize a composition including a medium that embodies a lotion, a salve, a cream, a paste, a slurry, an aerosol, a pad and a wrap. Additionally, the method can further utilize a medium containing an antimicrobial. Suitable antimicrobials include, but are not limited to an antibiotic, an antifungal, an antiviral and an antiseptic. The method can also utilize a medium including a topical anesthetic such as acetaminophen, ibuprofen, lidocaine, bupivacaine, prilocaine, and aloe vera. Finally, certain embodiments of the method can utilize a medium including an anti-inflammatory such as an NSAID, an omega-3 fatty acid, an extract of white willow bark, curcumin, ginger, an extract of green tea, pycnogenol, Boswellia serrata resin, resveratrol, Uncaria tomentosa, and capsaicin.

More specific embodiments of the methods and compositions disclosed include the following. A first aspect of the current disclosure includes a method for debriding damaged tissue from a wound, disease, or other condition requiring the removal of dead tissue. The method involves applying a fluid medium including an immobilized protease enzyme to the wound where the immobilized enzyme substantially resists degradation or deactivation in the wound and debrides damaged tissue contacted. The medium utilized can be a lotion, a paste, a cream, a slurry, and/or a salve. One embodiment of the composition includes an enzyme that maintains at least 50% of its initial activity after storage for at least about 4 months at temperatures from −20° C. to +50° C. Damaged tissue that can be debrided with embodiments of the compositions are caused by thermal energy, electrical energy, chemical exposure, or radiant energy or radiation. Specific forms of damaged tissue that can be treated by the composition include, but are not limited to a chronic wound, a pressure sore, gangrene, elevated MMP concentrations, a diabetic ulcer, pressure ulcer, a varicose ulcer, infection, a postoperative wound, a burn, a carbuncle, and a pilonidal cyst wound. Some embodiments of the method can be carried out with the medium contained on a material including, but not limited to a pad or a compress.

An additional aspect of the method disclosed includes applying a medium that includes an antimicrobial. Suitable antimicrobials include, but are not limited to an antibiotic, an antifungal, an antiviral and an antiseptic. Additionally, some embodiments applied can involve a medium further including a topical analgesic or anesthetic selected from the group consisting of acetaminophen, ibuprofen, lidocaine, bupivacaine, prilocaine, and aloe vera. Still further aspects of the method disclosed can involve the application of a medium including an anti-inflammatory agent selected from the group including an NSAID, omega-3 fatty acid, an extract of white willow bark, curcumin, ginger, an extract of green tea, pycnogenol, Boswellia serrata resin, resveratrol, Uncaria tomentosa, and capsaicin.

A further aspect of the current disclosure includes a composition that debrides damaged tissue from a wound, disease, or other condition that generates dead or dying tissue. One embodiment of the composition involves a fluid medium that includes an immobilized protease enzyme, where the immobilized protease enzyme substantially resists degradation or deactivation in the wound and is stable to temperatures of at least 25° C. Certain embodiments of the composition include an immobilized enzyme that maintains at least 50% of its initial activity after storage for at least 4 months at temperatures from −20° C. to +50° C. A further aspect of the composition includes an enzyme that is not immobilized, is immobilized on the same support as the protease enzyme, or is immobilized on a second support. Certain embodiments of the composition involve a medium selected from the group consisting of a lotion, a salve, a cream, a slurry, and a paste. For certain compositions, it is useful to utilize the fluid medium applied to a pad or compress.

Further embodiments disclosed herein include compositions where the fluid medium includes an antimicrobial selected from the group consisting of an antibiotic, an antifungal, an antiviral and an antiseptic. Still further embodiments of the composition include, but are not limited to, a topical analgesic or anesthetic. Even further embodiments of the current disclosure involve compositions that include a topical analgesic or anesthetic selected from the group consisting of acetaminophen, ibuprofen, lidocaine, bupivacaine, prilocaine, and aloe vera. Still further embodiments of this disclosure include an anti-inflammatory agent selected from the group consisting of NSAID's, omega-3 fatty acid, extract of white willow bark, curcumin, ginger, extract of green tea, pycnogenol, Boswellia serrata resin, resveratrol, Uncaria tomentosa, and capsaicin.

Finally, a further aspect of the current disclosure involves a composition for debriding damaged tissue from a wound, condition or disease. The composition includes, but is not limited to, a fluid medium further including an immobilized protease enzyme derived from a protease enzyme, a crosslinker, and at least one polyfunctional material. The composition's fluid medium can include a lotion, a salve, a cream, a slurry, and a paste. Upon application to the damage tissue, the composition debrides damaged tissue contacted.

### DESCRIPTION

The current disclosure involves a medicant and methods for its use in the treatment of wounds and the debridement of dead tissue resulting from burns (including chemical burns) and related injuries or conditions that require the removal of dead and injured tissue. Treatments are suitable for human use as well as other mammals or animals provided the normal procedures required for treating those mammals for tissue damage are followed.

Different embodiments of the medicants disclosed herein involve at least one active component that:
is immobilized into a solid support;
degrades the dead/injured tissue without diffusing into the wound;
reduces the excess proteolytic activity of MMPs
exhibits sufficient stability to remain effective in the field without refrigeration and improved stability in high temperature applications;
is included in or on a vehicle suitable for application to a wound;
improves pH stability, and
reduces risk of damage to healthy tissue.

Although enzymes studied thus far have included proteolytic enzymes including bromelain, collagenase and trypsin, suitable enzymes are not so limited. Initial support materials for these enzymes have included, but are not limited to, support materials taught in Applicant's pending U.S. patent application Ser. No. 15/789,941, and published as US 2018/0110868 A1. Applicant's earlier patent application is hereby incorporated by reference herein. A further feature of the medicants developed herein includes their resistance to autocatalytic degradation. Some embodiments may include multiple enzymes immobilized, together or separately, and incorporated into a delivery vehicle (lotion, creams, salves, pastes, slurries, wound pad or dressing).

Certain embodiments of the medicant disclosed include one or more antimicrobials to prevent infection during the debridement and healing process. The only limitations for the antimicrobials other than suitability and efficacy toward the wound being treated include stability under storage and treatment conditions and compatibility with other components of the medicant. In some embodiments, the antimicrobial could include enzyme, immobilized or not, that have antimicrobial effects such as lysozyme. Antimicrobials considered thus far are disclosed below.

In the studies described in the text, several covalently bonded immobilized enzymes and related medicants were prepared, and the following issues are described in detail:
the effect of immobilization and enzyme selection on the resulting enzyme activity;
the immobilized enzyme's shelf life, heat stability, and resistance to autolysis,
efficacy of the immobilized enzyme in degrading artificial wound eschar (AWE);
the compatibility of several antimicrobials with the immobilized enzymes;
the dermal compatibility of the different formulations;
the ability of the immobilized enzyme to resist degradation by MMPs present in the wound, while at the same time deactivating those MMPs; and
the comparison of the efficacy of the novel formulations with regard to commercial products designed for enzymatic debridement.

### Immobilized Enzymes for Wound Debridement and Wound Treatment:

### Introductory Comments:

The immobilization process chemically bonds active enzymes to inactive and biocompatible support materials. Proper immobilization of the enzymes 1) enables protections from heat-degradation, 2) prevents autolytic action improving shelf life and enzyme life during application, and 3) protects the enzyme from other harmful environments such as denaturing, acidic, or caustic conditions. Support material selection is driven by compatibility requirements for the specific reaction environment. An additional advantage of this process is that certain embodiments utilize low-cost and common materials such as chitosan, and/or repurposed byproducts from other biological manufacturing processes such as fungal mycelium, bacterial cell components, or lignocellulosic matter.

### Immobilization Methods:

Separate immobilized enzymes including bromelain, collagenase, and trypsin are prepared based on the procedures described below and in Applicant's pending U.S. patent application Ser. No. 15/789,941, and published as US 2018/0110868 A1. The formulations include an enzyme, one or two support matrix materials, and a crosslinker. The baseline formulation for each enzyme series consists of 40-60 wt. % enzyme in 10% increments and 20 wt. % low-molecular weight chitosan. The remainder of the formulation is made up of a secondary support material and crosslinker. The secondary support materials in the formulation provide additional structure and material processing enhancements to the final product, which will vary depending on the nature of the enzyme used in the formulation. Secondary support materials utilized include maltodextrin, gum Arabic, dried and ground tomato pumice, A. niger mycelium, and tetramethylbenzidine.

Crosslinking of the components is achieved through amine-based reactions. Two types of crosslinkers were used, dialdehydes and organic diacids. Different chain lengths between the paired functional groups were considered with regard to effects on stability and activity of the immobilized enzyme. After the components were crosslinked, the immobilized samples were dried by: spray drying (SD), freeze drying (FD), or fluid bed drying. Following drying, the immobilized enzymes can be passed through a screen to ensure particle size uniformity, for example less than 100 μm. Powders that do not pass through the screen were ground via ball mill until they pass.

### Testing Procedures:

### Enzyme Activity Measurements:

Immobilized enzymes were compared to their natural counterparts (the parent enzymes) using a universal protease assay, with casein or azocasein as a substrate. Using the same assay for all proteases allowed for their performance to be compared directly to one another early in production. For each test a given amount of enzyme was added to the substrate solution, mixed and incubated at 37° C. for 10 min. Following the incubation, trichloroacetic acid was added to the solution to stop the reaction and incubated for another 30 min. Following this second incubation, test solutions were filtered using a filter having a 0.45 μm mesh to remove insoluble material. Sodium carbonate and colorant reagent were added to the filtered solutions, and then they were incubated for another 30 min at 37° C. At the end of this incubation period samples were measured for absorbance at 660 nm, where the total absorbance corresponds to the activity of the enzymes. Natural enzymes were used to establish standard curves with respect to enzyme mass.

### Long-Term Stability at Different Temperatures:

Each candidate immobilized enzyme powder produced was distributed into amber screwcap vials and placed under a storage test condition (−20° C., 4° C., 25° C., and 50° C.) for long-term stability testing. Samples were evaluated every 2 weeks for the first 2 months and then monthly up to 52 weeks of storage. At each testing point, 3 of the 4 stored vials were selected and 3-5 samples were taken from each vial for measurement using the activity assay. Sampled vials were returned to storage until the next time point. Results of the activity measurements were averaged for each immobilized enzyme and compared to each sample's initial recorded activity value.

The retained activities±standard error of various tested materials at 4, 8, and 12 months are listed in Table 1. FD refers to immobilized enzyme prepared with freeze drying and SD refers to immobilized enzyme prepared using spray drying. The FD Collagenase material stands out as its measurable activity effectively did not change over the course of the test at any of the tested storage conditions. Overall, samples stored at −20° C. retained most of their activity through the course of the test and those stored at 50° C. lost the most activity. The 4° C. and 25° C. samples generally had similar activities over the course of testing.

TABLE 1
Retained Activity of Immobilized Enzymes after Various Times of Dry Storage

| | months | 50° C. | 25° C. | 4° C. | −20° C. |
|---|---|---|---|---|---|
| FD Bromelain: | 4 | 63.9% ± 2.4% | 95.6% ± 3.3% | 96.0% ± 4.0% | 100.4% ± 3.1% |
| | 8 | 45.9% ± 2.2% | 54.0% ± 4.0% | 55.5% ± 1.3% | 92.7% ± 3.6% |
| | 12 | 44.5% ± 1.0% | 58.9% ± 2.9% | 53.8% ± 2.6% | 95.2% ± 4.0% |
| SD Bromelain: | 4 | 69.5% ± 1.4% | 77.9% ± 2.6% | 69.2% ± 2.1% | 100.1% ± 4.4% |
| | 8 | 71.2% ± 0.7% | 58.7% ± 0.9% | 56.8% ± 0.9% | 84.7% ± 1.9% |
| | 12 | 45.0% ± 1.0% | 67.6% ± 0.8% | 71.6% ± 0.8% | 87.2% ± 1.0% |
| FD Collagenase: | 4 | 100.0% ± 6.4% | 103.5% ± 3.6% | 102.3% ± 3.9% | 99.7% ± 2.6% |
| | 8 | 96.7% ± 2.4% | 98.9% ± 5.7% | 95.2% ± 2.0% | 98.9% ± 2.2% |
| | 12 | 101.2% ± 1.8% | 98.3% ± 5.1% | 102.8% ± 2.7% | 99.0% ± 5.4% |
| SD: Collagenase: | 4 | 67.7% ± 1.7% | 102.0% ± 4.2% | 101.7% ± 1.0% | 100.7% ± 1.2% |
| | 8 | 36.4% ± 1.8% | 94.4% ± 2.7% | 78.5% ± 1.9% | 101.5% ± 1.2% |
| | 12 | 39.7% ± 1.6% | 56.6% ± 0.6% | 70.0% ± 2.4% | 85.3% ± 1.9% |
| FD Trypsin: | 4 | 80.7% ± 1.1% | 101.4% ± 3.1% | 100.7% ± 3.4% | 102.0% ± 2.4% |
| | 8 | 70.4% ± 1.2% | 89.3% ± 3.9% | 56.2% ± 2.2% | 90.4% ± 0.9% |
| | 12 | 55.7% ± 1.5% | 88.4% ± 1.7% | 66.5% ± 2.2% | 101.8% ± 1.1% |
| SD Trypsin: | 4 | 62.0% ± 1.1% | 93.9% ± 1.7% | 96.5% ± 1.7% | 100.8% ± 3.4% |
| | 8 | 46.7% ± 1.2% | 71.0% ± 1.5% | 85.9% ± 6.0% | 97.9% ± 1.8% |
| | 12 | 44.4% ± 1.0% | 62.1% ± 1.2% | 56.5% ± 2.1% | 83.3% ± 1.6% |

### pH Stability and Autolytic Resistance:

Natural (non-immobilized) and immobilized forms of each enzyme were loaded into 10 mL of phosphate buffer solutions at pH 7.0, 6.0, and 5.0 and mixed for 6 hours at 37° C. These pH values were selected as representative of the range of pH of intact to wounded skin. The amount of natural or immobilized enzyme material that was added at the start of the test was based on its measured activity, so that all tests started with similar enzyme activities for each enzyme: 2.6 U bromelain, 21 U collagenase, and 5.4 U trypsin. Retained activity was determined by taking a sample of the test solution at a given time point and evaluating it against casein. The measured activity of the test sample was divided by the activity measured at the start of the test and is presented as the average of 5 measurements±standard error, Table 2.

TABLE 2
Retained activity of natural and immobilized enzymes
after 6 hours of exposure to different pH conditions.

| | | pH 5 | pH 6 | pH 7 |
|---|---|---|---|---|
| bromelain | Natural | 95.6% ± 4.9% | 36.1% ± 1.4% | 64.7% ± 3.0% |
| | Immobilized - Freeze Dried | 100.0% ± 1.0% | 63.4% ± 3.4% | 74.9% ± 2.4% |
| | Immobilized - Spray Dried | 100.0% ± 1.8% | 54.1% ± 3.6% | 73.7% ± 3.2% |
| collagenase | Natural | 12.3% ± 4.5% | 18.9% ± 1.3% | 13.9% ± 3.3% |
| | Immobilized - Freeze Dried | 44.1% ± 2.6% | 31.6% ± 2.4% | 24.5% ± 2.4% |
| | Immobilized - Spray Dried | 35.9% ± 1.6% | 60.2% ± 5.1% | 19.4% ± 1.4% |
| trypsin | Natural | 43.3% ± 1.1% | 46.1% ± 1.2% | 7.7% ± 5.3% |
| | Immobilized - Freeze Dried | 46.8% ± 1.7% | 65.2% ± 2.6% | 94.1% ± 1.1% |
| | Immobilized - Spray Dried | 77.0% ± 1.6% | 65.9% ± 3.0% | 81.9% ± 3.5% |

Bromelain was very-self stable in its natural form at a pH of 5, and this is matched by its immobilized forms. As the pH is raised the natural version became susceptible to autolytic degradation at a pH of 6 and appeared somewhat more stable again at a pH 7. This is mirrored by its immobilized forms, although both immobilizations retain more of their original activity than the natural form. Collagenase lost 80-90% of its initial activity within 6 hours while in its natural form at all pH tested. While the immobilized forms also showed a loss of enzyme activity with exposure, they generally retain 2× to 3× more activity at the end of the test. Natural trypsin performed as expected, losing most of its activity (92% of the starting activity) at pH 7, where it is most active. At more acidic pHs natural trypsin does not have as much activity, and as such does not self-degrade nearly as much after 6 hours. The immobilized trypsin candidates showed improved activity retention vs. natural enzyme at all the tested pHs, with the best activity retention at a pH of 7. In general, these testing results validated the hypothesis that immobilizing the enzymes grants them extended resistance to autolysis. These results suggest that smaller concentrations of immobilized enzyme may be more effective for a longer amount of time when applied in a final treatment formulation.

Performance Evaluation with Simulated Necrotic Tissue:

Testing of the immobilized enzymes was performed on an artificial wound eschar (AWE) membrane consisting of 65% collagen, 10% elastin, 10% fibrin, and 15% fibrinogen by weight. The three proteins were each labeled with a different fluorescent dye: collagen with fluorescein isothiocyanate, elastin with rhodamine, and fibrin with coumarin. The AWE was prepared by mixing the labeled proteins in 10 mL of 50 mM tris buffer at pH 7.4 for 5 minutes with a tissue tearer. 10 mL of buffer containing the fibrinogen was mixed into the labeled proteins. 50 U/mL of thrombin was then quickly mixed in to begin the coagulation process. The mixture is poured out into 25 mm diameter Teflon sample holders backed by a pre-cut nylon support membrane. The membrane was covered, and the proteins coagulate at room temperature for 30 minutes. The membranes were then gently washed with DI water and stored at 4° C. for at least 24 hours before testing.

For testing, an AWE membrane is placed on a Franz cell containing 15 mL of Tris buffer at pH 7.4 with 1% penicillin-streptomycin in its receptor. The temperature of the cell was brought to 35° C. and held there for the duration of the test. At time=0, a 0.4 mL volume of buffer containing immobilized or natural enzyme at concentrations between 0.25-8.50 mg/mL was added to the top of the membrane and the top covered. As the enzyme degrades the membrane, fluorescent dyes are released into the receptor chamber, which was regularly sampled and tested for the presence of the dyes over a 24-hour period. The intensity of the fluorescent response was proportional to the concentration of each digested protein.

The fluorescent intensity for each protein was measured at each time point. The concentration of protein in the receptor was calculated through a calibration curve for each labeled protein. This result was then divided by the total amount of each protein present in each AWE to indicate how much of each protein in the AWE had been broken down over time. Generally, all the immobilized enzymes demonstrated the most proteolytic activity during the first 6-8 hours of the test, which would then transition to a slower rate of degradation for the remainder of the test. The time of the transition between the two degradation rates was dependent on the concentration of immobilized enzyme in the test with higher test concentrations producing higher fluorescent intensities and transitioning to the slower rate at earlier time points. Lower doses of immobilized enzyme (<2 mg/mL) did not show much difference in degradation rates.

At the end of each experiment the AWE substrate's appearance was examined. Immobilized collagenase demonstrated the most visually complete digestion of the AWE membrane for all tested dosing, the membrane was significantly degraded with little material being left on the membrane. The same degree of membrane degradation was not visible for the tests with immobilized bromelain and trypsin, although visually the membranes were melted or gel-like at the end of the test with a faded color when compared to both the membrane pre-reaction and to controls without enzyme. This difference in the final membrane state between the immobilized enzymes is due to the activity per mg of the immobilized collagenases being 3-5 times higher than the activity of the other immobilized samples, and because collagen is the natural substrate of collagenase it is inherently more reactive towards the AWE.

The degradation of individual protein concentrations can be combined to give a measurement of the total degradation of the AWE membrane over time. The slope of the initial linear portion can be determined to give a maximum degradation rate which can then be divided by the activity of the enzyme with respect to casein and averaged. This provides a direct method to compare the performance of the immobilized enzyme materials to one another, Table 3.

TABLE 3
Comparison of Immobilized Material AWE-lytic Performance

| | μg AWE/hr/mg | μg AWE/hr/Unit |
|---|---|---|
| | Immobilized Enzyme | Activity (casein) |
| FD Bromelain | 15,945 ± 2,516 | 3,872 ± 611 |
| SD Bromelain | 18,243 ± 7,723 | 4,258 ± 1,802 |
| FD Collagenase | 21,238 ± 4,355 | 18,571 ± 3,808 |
| SD Collagenase | 20,447 ± 4,432 | 27,225 ± 5,901 |
| FD Trypsin | 12,125 ± 2,499 | 3,437 ± 708 |
| SD Trypsin | 10,456 ± 1,136 | 3,318 ± 361 |

Compatibility with Common Antimicrobials:

Stock solutions of individual antimicrobials were prepared at the following concentrations: silver sulfadiazine at 4% wt./vol., cerium nitrate at 1M, and extracted Neosporin® at 10% wt./vol. For Neosporin®, the antibiotics were extracted from the Vaseline carrier by mixing the appropriate mass of material in buffer, heating to 35-37° C. while mixing to such that the Vaseline liquefies, and the antibiotics diffuse to the aqueous phase. Control tests were performed and verified no growth for both bacteria at these concentrations. To test antimicrobial compatibility with the immobilized enzyme, 40 mg of an immobilized enzyme material was added to 2 mL of the antimicrobial stock solution. The solution was mixed at room temperature and 100 μL samples were taken at 4 hours and 24 hours to be tested for antimicrobial efficacy. Each 200 μL sample is mixed with 2 mL of a bacterial culture, S. aureus (ATCC 25923) or P. aeruginosa (ATCC 27853), at an OD600 of approximately 0.3. This mixture was incubated for 2 hours and then 100 μL was sampled and spread on a culture plate. The plates were incubated at 37° C. overnight and the colonies were counted the following day.

Immobilized enzyme candidates were evaluated at concentrations of 1-3 mg/mL with previously prepared antimicrobial stock solutions. Samples were taken from each mixture at 24 hours to evaluate the remaining enzyme activity. To reduce issues of signal interference on the spectrophotometer, the samples were diluted and compared to controls with no enzyme. Additional tests were performed to evaluate the performance of immobilized enzyme exposed to 100 μM concentrations of copper-Il-chloride and iron-Il-sulfate to determine if metal ions naturally present in blood and interstitial fluid would interfere with enzyme performance.

The results from mixing the antimicrobials and immobilized enzyme are shown in Table 4. In general, the FD samples demonstrated a better performance, indicated by lower CFU counts, than the SD ones in all cases against both bacteria. Against S. aureus mixing with FD samples at 4 hours there were less than 10 CFU/plate, and at 24 hours only the cerium nitrate showed a notable degradation in performance with 94 CFU for the FD Bromelain sample and roughly 25 CFU for the other two FD enzymes. Only Neosporin® demonstrated any useful compatibility with all the SD immobilized enzymes, although silver sulfadiazine mixed with SD Bromalin produced only 25 CFU after 24 hours. The results of testing with P. aeruginosa show similar performance with the FD immobilized enzymes having less derogatory effects on the antibiotics than the SD immobilized enzymes. In this case the silver sulfadiazine was seen to be more effective against the bacteria when combined with all the enzymes followed by the cerium nitrate, then the Neosporin®. Test results show that the FD immobilized enzymes are significantly more compatible with the three selected antibiotics than their SD counterparts. Of the tested antimicrobials the FD immobilized enzymes show only a minimal effect on the performance of silver sulfadiazine and similarly with Neosporin®

TABLE 4
Antimicrobial Stability in the Presence of Immobilized Enzymes

| | | | Silver | Cerium | |
|---|---|---|---|---|---|
| | Exposure | | Sulfadiazine | Nitrate | Neosporin ® |
| Bacteria | Time [hr] | Enzyme | [CFU] | [CFU] | [CFU] |
| S. aureus | 4 | FD Bromelain | 1 | 0 | 0 |
| | | SD Bromelain | 15 | >200 | 0 |
| | | FD Collagenase | 5 | 0 | 0 |
| | | SD Collagenase | 183 | 174 | 1 |
| | | FD Trypsin | 0 | 0 | 0 |
| | | SD Trypsin | >200 | 4 | 1 |
| | 24 | FD Bromelain | 0 | 94 | 0 |
| | | SD Bromelain | 21 | >200 | 0 |
| | | FD Collagenase | 1 | 25 | 3 |
| | | SD Collagenase | >200 | >200 | 40 |
| | | FD Trypsin | 0 | 27 | 0 |
| | | SD Trypsin | >200 | >200 | 10 |
| P. aeruginosa | 4 | FD Bromelain | 0 | 0 | 1 |
| | | SD Bromelain | 0 | >200 | >200 |
| | | FD Collagenase | 0 | 0 | 4 |
| | | SD Collagenase | 71 | >200 | >200 |
| | | FD Trypsin | 0 | 0 | 1 |
| | | SD Trypsin | 2 | 0 | >200 |
| | 24 | FD Bromelain | 0 | >200 | 15 |
| | | SD Bromelain | 7 | >200 | >200 |
| | | FD Collagenase | 0 | >200 | >200 |
| | | SD Collagenase | 70 | >200 | >200 |
| | | FD Trypsin | 0 | 0 | 13 |
| | | SD Trypsin | >200 | 42 | >200 |

where only the FD collagenase shows significant growth with P. aeruginosa at 24 hours. Cerium nitrate appears to be the most affected by the presence of the immobilized enzymes, losing all its protective activity when mixed with SD Bromelain and SD Collagenase. Control tests with only the antibiotic solutions verified they were effective against both bacteria at the tested concentrations, producing plates with no colonies. Controls with only immobilized enzyme; however, showed no measurable effect at stopping bacterial growth at the concentrations used for this testing developing full plates by 4 hours.

The results testing retained enzyme activity are more varied by enzyme and antimicrobial than what was seen in the tests of the degradation of antimicrobial capability, Table 5. The retention of activity in the presence of antimicrobials can be looked at in two different ways. Considering the data by antimicrobial, Neosporin® has the least overall effect on the activity of the enzymes, while silver sulfadiazine has the most significant effect—reducing the activity of 3 of the 6 candidates to less than 40%

TABLE 5
Retained Activity After Exposure to Antimicrobials and Metal Ions for 24 Hours

| | Silver | Cerium | | Iron-II- | Copper-II- |
|---|---|---|---|---|---|
| Enzyme | Sulfadiazine | Nitrate | Neosporin ® | Sulfate | Chloride |
| FD Bromelain | 57.8% ± 4.1% | 81.4% ± 25.8% | 66.8% ± 4.5% | 96.3% ± 6.8% | 71.9% ± 6.3% |
| SD Bromelain | 97.9% ± 9.2% | 87.8% ± 15.4% | 91.4% ± 4.9% | 86.7% ± 1.6% | 86.6% ± 3.2% |
| FD Collagenase | 23.5% ± 2.7% | 34.2% ± 6.2% | 79.7% ± 6.5% | 72.3% ± 1.0% | 76.2% ± 4.3% |
| SD Collagenase | 23.7% ± 3.9% | 26.1% ± 5.8% | 67.7% ± 4.0% | 79.6% ± 0.3% | 82.9% ± 1.9% |
| FD Trypsin | 38.6% ± 8.2% | 78.5% ± 3.7% | 64.0% ± 7.7% | 80.0% ± 6.3% | 71.6% ± 1.2% |
| SD Trypsin | 63.3% ± 13.2% | 40.2% ± 14.4% | 57.4% ± 11.7% | 79.8% ± 1.9% | 93.0% ± 1.9% |

of their initial activity after 24 hours. Considering the data from a broad enzyme compatibility perspective, the immobilized enzyme most compatible across the tested antibiotics is SD Bromelain, followed by FD Bromelain. The trypsin's' performance is next with the FD Trypsin showing resistance to degradation against cerium nitrate and Neosporin®, while the SD Trypsin was more resistant to silver sulfadiazine. The Collagenases had the lowest retained activities, with less than 35% retained after exposure to cerium nitrate and silver sulfadiazine.

### Evaluation of In Vitro Dermal Compatibility:

Strat-M™ membranes (EMD Millipore) were selected and for testing diffusion of the immobilized enzyme through healthy skin. Strat-M is a synthetic polymeric membrane designed to simulate the properties of human skin for transdermal diffusion. The membrane is composed of multiple polyether sulfone layers with a tightly packed surface layer to create a morphology that resembles human skin. The advantage of this material is that it provides a more predictive result, since it has less variability between samples, and is not as limited to storage conditions, safety considerations, and sample availability.

Franz cells with 5 mL receptor volumes were filled with filtered 10 mM PBS buffer at pH 7.4 and held at 37° C. Strat-M membranes were placed on the receptor, covered with 0.3 mL of buffer, and the system was allowed to equilibrate with the receptor fluid for at least 30 minutes. After equilibration 0.5 mL of solutions containing 20-100 mg/mL of natural enzyme, immobilized enzyme, or BSA as a control were added to the top of the Franz cell and the cell was covered. 0.5 mL samples were taken hourly (replacing the volume with fresh PBS buffer each time) for the first 6 hours and a final sample at 24 hours. The protein concentration in each sample was evaluated using a Bradford assay and a 660 nm protein concentration assay from Pierce. The enzyme activity of the 24-hour samples was evaluated to determine if active enzyme or just peptides had passed through the membrane.

Tests of the immobilized enzymes were performed to compare the diffusive breakthrough with their natural enzyme counterparts. Samples from all three natural enzymes demonstrated enzyme activity verifying that intact enzyme had crossed through the membrane, although the activity calculated by the testing indicated that most of the material that had passed through the membrane in the case of trypsin and bromelain was degraded or inactive enzyme. While bromelain's measured activity was closer to the measured concentration of protein in the sample. Like the natural enzymes, the loading of the immobilized enzyme in the Franz cell did not significantly change the amount of protein measured in the receptor. The change in protein concentration with time followed the same pattern of flattening out between the 6-hour and 24-hour measurements. The amount of protein in the receptor chamber of the Franz cell averaged across all tests at the 6-hour time point was used as the primary comparison point for each sample as it appears to be close to where each material transitions from a rapid change in concentration to a slower one Table 6. Both FD and SD versions of each enzyme immobilization perform similarly to one another in terms of the amount of protein crossing the membrane. The immobilized bromelain has lowest protein crossover similar to its natural version, followed by the immobilized collagenase and then immobilized trypsin.

TABLE 6
Amount of Protein Passing Through Strat-M ™ Membranes
After 6 Hours

| Enzyme | Protein [mg] |
|---|---|
| Bromelain | 2.08 ± 0.25 |
| FD Bromelain | 0.10 ± 0.02 |
| SD Bromelain | 0.62 ± 0.27 |
| Collagenase | 18.15 ± 0.81 |
| FD Collagenase | 1.30 ± 0.25 |
| SD Collagenase | 1.37 ± 0.53 |
| Trypsin | 11.93 ± 0.50 |
| FD Trypsin | 5.80 ± 0.42 |
| SD Trypsin | 7.36 ± 0.89 |

Tests for proteolytic activity of the final receptor fluids for the immobilized enzyme samples did not show a measurable reaction for the immobilized enzymes. This implies that what made it through the membrane was more peptide in nature than enzyme. This may also be why the immobilized trypsin measurement is so much higher as it could be the case that the peptides that did get separated off the immobilization matrix happened to be more responsive to the marker dye for measuring protein concentration.

Immobilized enzyme samples were further analyzed to determine if they have a detrimental effect on healthy tissue surrounding a burn wound. Human keratinocytes and fibroblasts obtained from ATCC, were seeded at cell densities of 2×10⁴ in 24 well plates 6 days prior to experiments and incubated at 37° C. under 5% CO₂. On day 7 a stock solution of 100 mg/mL an immobilized enzyme or natural enzyme is prepared in either PBS or a culture medium. Cells were treated with 500 μL of the enzyme solution, at several dilutions, for up to 6 hours. At the end of the test, the enzyme solution is removed, cells were washed twice with PBS, and 500 μL of the appropriate cultivation media is added. The effect of the different concentrations of enzyme on the cells is assessed using a resazurine-based assay and life cell imaging. In this case the fluorescence signal is proportional to the amount of vital cells. All enzyme concentrations were measured in triplicate (n=3). The enzyme containing fluid removed after 6 hours of exposure to cells is evaluated using the methods described above for enzymatic activity. Data determined above is used to compensate for autolytic degradation in test samples.

For the fibroblast compatibility study natural bromelain shows a small beneficial effect on growth at all 4 tested concentrations, with an average relative signal between 2.0 and 3.0. The freeze-dried bromelain (FDB) at the lower concentrations has no net effect but starts to show a detrimental effect as its concentration increases. The spray dried bromelain (SDB) appears to be the most beneficial of the three bromelains tested with a relative signal >3.5 at 60 and 80 mg/mL. The relative signal of the natural collagenase mixed cells remained close to 1 for all concentrations. The freeze-dried collagenase (FDC) started out at no net effect, at concentrations of 60 and 80 mg/mL it produced 10 times the signal for the first hour which steadily decreased over the next 2 hours. The spray dried collagenase (SDC) showed a similar pattern of performance with the 20 mg/mL concentration producing 4 times the signal for the 1st-3rd hours of testing. At higher concentrations the 1-hour signal increased with increasing concentration; however, it would then decrease over time with the 80 mg/mL test sample returning to a relative signal of 4 at the end of 3 hours. The natural trypsin had a similar performance to the collagenase, showing almost no change to cell growth at all tested concentrations. The freeze-dried trypsin (FDT) showed some improvement in cell growth. The relative signal slightly increased over time for all samples tests, with the highest signal (˜2.25) occurring at 80 mg/mL after 3 hours. Similarly, to the other spray dried enzymes, the spray dried trypsin (SDT) performed the best of the three samples, with the relative signal again increasing with test concentration. However, it appears to peak after two hours of exposure with a slow decrease following thereafter.

The immobilized enzymes showed comparable results with the keratinocyte testing in terms of the overall pattern, but in all cases where there was an improved growth signal it was significantly higher than what was seen in testing the fibroblasts. Natural bromelain produced an increasing signal with time at all tested concentrations, the highest relative signal (6.71) occurring at 40 mg/mL. The relative signal for FDB remained around 1 at all tested concentrations, while the relative signal for SDB started at 6.87 after 1 hour at 20 mg/mL. Subsequent SDB concentrations produced higher 1-hour signals, up to 12.40 at 80 mg/mL. At all SDB concentrations the signal would increase for the 2-hour reading and at 3 hours it would have declined below the 1-hour reading, but still be higher than a value of 1. Again, collagenase had the most impressive performance of the tested enzymes. In each tested case the relative signal started higher than 2, increased for the 2-hour measurement, and decreased for the 3-hour measurement. The increase in signal was proportional to the increase in enzyme concentration with FDC at 80 mg/mL showing a relative signal on the order of 60. The natural trypsin performed similarly to before with relative signals close to 1 for all concentrations and times. Similarly, FDT and SDT followed the same patterns here as they did with fibroblasts. With the SDT showing a peak relative signal after 2 hours at 80 mg/mL.

For determining the efficacy of immobilized enzymes with respect to MMPs, immobilized enzymes were stirred in PBS solutions at pH 7.5 and 30° C., also containing one of the following MMPs: MMP-2, MMP-8, or MMP-9. The reaction solution is stirred to ensure the interaction of the MMPs with the immobilized enzymes in the container preventing an artificially low measurement. The mixture reacts for one hour and then is sampled and the final MMP concentration measured. MMP concentrations were measured by ELISA and complimented by zymography of the samples utilizing SDS-PAGE methods under denaturing, non-reducing conditions in gels containing the appropriate substrates (gelatin, casein, collagen) for the MMP being analyzed. Together these tests confirm the efficacy of the immobilized enzyme for reducing MMP concentrations in a wound environment. Controls consisting of only MMP or immobilized enzyme were used to confirm that self-lytic action is not responsible for degradation and to reduce potential signal interference from complex mixtures. In addition to measuring the concentrations of MMP, the activity of the immobilized enzyme was monitored for changes in activity over the course of a reaction.

### Lotion Performance:

To evaluate how the immobilized enzyme perform in a product-like form, a simple ointment was prepared by blending the best performing immobilized enzymes, as identified by prior testing, using standard ingredients at a similar enzyme activity loading to current commercial products. Additional fluids were prepared that contain natural enzymes at the same activity loading, and fluids that combine the best performing immobilized enzyme forms of bromelain, collagenase, and trypsin to identify synergistic performances. Immobilized enzyme lotions were initially evaluated against AWE using the Franz diffusion cell system and methods described above to determine performance. Samples of the commercial products were also evaluated as commercial controls. Samples of immobilized enzyme-based lotions, natural enzyme lotions, and commercial samples were stored in sealed, light-proof containers at 25° C. and 50° C. for up to one month as an evaluation of storage stability.

The results from this testing demonstrated that the immobilized enzyme materials in lotion maintained a similar performance profile to earlier testing. Visual inspection of the AWE membrane degradation at the conclusion of the tests revealed that the membranes exposed to several of the candidate immobilized enzymes were more broken down than the ones exposed to Santyl. Analysis of the tagged elastin, collagen, and fibrin accumulation in the receptor fluid showed that all the enzyme materials performed in a similar pattern to prior testing. The test started with a rapid initial degradation of the AWE that over the course of the first 5 hours slowed to, on average, 10% of the initial rate. The elastin and collagen content in the receptor fluid was proportional to its loading in the membrane suggesting that one was not preferentially released over the other. This was not the case for the fibrin, because not all the fibrin present in an AWE membrane is tagged with a florescent marker. The samples were compared by the rate of AWE digestion in terms of mg detected protein/hour/g lotion for the rapid period, the slower period, and the total amount of protein detected at the end of the test (˜24 hours). The FDB, and FDT in lotion performed the best vs. Santyl in terms of rate of AWE degradation, exceeding Santyl by a factor of 10%. FDC in lotion along with FDT, SDB, and SDT in the modified Zn lotion all had similar AWE degradation rate profiles to Santyl. While the remaining immobilized enzyme materials had degradation rates <90% of Santyl's measured rate using this test.

### Preparation of Specific Discussed Materials:

### Example 1: Immobilized Enzyme Production Followed by Spray Drying

7-8 g chitosan and 7-8 mL of acetic acid were mixed in 400 mL of DI water until solubilized. 20-25 g of enzyme, 5.5 g dried A. niger mycelium, and 5.5 g gum Arabic were then added to the solution and stirred until homogenous, then 0.8 g of terephthalaldehyde was slowly added to crosslink the reactants. The solution was allowed to mix for 10 minutes, and then was fed into a Yamato ADL311S lab spray dryer at 2.5 mL/min. The inlet temperature of the spray dryer was set at 110° C. and the atomizing air and blower values were adjusted to maintain the formation of powder and an outlet temperature >40° C. Dried materials were collected, checked for particle size by sieving, and stored sealed at 4° C. until tested.

### Example 2: Immobilized Enzyme Production Followed by Freeze Drying

4.0 g chitosan was mixed with 4-5 mL of acetic acid in 325 mL of DI water until the chitosan had solubilized. 3 g of tetramethylbenzidine and 3 g of dried A. niger mycelium were added and stirred into solution. 10-15 g of enzyme was then added and the solution was mixed for 15 min. 0.85 g of azelaic acid to was added initiate crosslinking and the solution was mixed for an addition 10 min before being frozen at −80° C. The frozen solutions were dried using a Labconco Freezone 1L BT. Materials remained on the freeze dryer for up to 48 hours. Dried immobilized enzyme were collected, checked for particle size by sieving, and stored in sealed containers at 4° C. until needed.

### Example 3: Immobilized Enzyme Preparation in a Cream

31.5 g of an oil/water emulsifier mixture (PEG-6 stearate, ethylene glycol stearate, and PEG-32 stearate), 15 g of surfactant (oleoyl polyoxyl-6 glycerides), 20.25 g mineral oil, 88.4 g DI water, and 0.15 g sorbic acid were mixed together and heated to 75° C. After 5 minutes at 75° C. the mixture was slowly cooled while being mixed at 350 RPM. After the mixture cools below 35° C., 3.75 g of powdered immobilized enzyme is added to the mixture. The cream continues to be mixed until it reaches room temperature, then it is separated into containers and stored at room temperature until tested.

### Example 4. Immobilized Enzyme Preparation in a Lotion

An oil phase solution consisting of 7.5 g of oil/water emulsifier mixture (PEG-6 stearate and PEG-32 stearate), 1.5 g of stearyl alcohol, and 22.5 g mineral oil was mixed and heated to 75° C. In parallel an aqueous solution consisting of 114.6 g DI water and 0.15 g sorbic acid was also mixed and heated to 75° C. Once both mixtures were at temperature, the aqueous phase was slowly poured into the oil phase while stirring. The complete lotion was mixed and cooled to 35° C., 3.75 g of powdered immobilized enzyme were added, and the lotion was stirred at 350 RPM while it cooled to room temperature. After cooling the lotion was separated into containers and stored at room temperature until testing.

While the disclosed technology has been illustrated, and described in detail in the foregoing description, the same is to be considered as illustrative and not restrictive in character. It is understood that the embodiments have been shown and described in the foregoing specification in satisfaction of the enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the disclosed technology are desired to be protected.

1. A method for debriding damaged tissue from a wound, disease, or other condition comprising applying a medium to the wound, the medium including an immobilized enzyme,
wherein the medium is a fluid medium,
wherein the immobilized enzyme is a protease enzyme,
wherein the immobilized enzyme substantially resists degradation or deactivation in the wound, and
wherein the immobilized enzyme debrides the damaged tissue contacted.

2. The method of claim 1 wherein applying a medium to the wound involves applying a medium to the wound having a form selected from the group consisting of a lotion, a paste, a cream, a slurry, a salve, and an aerosol.

3. The method of claim 1 wherein applying a medium to the wound involves applying a medium to the wound including an immobilized enzyme that maintains at least 50% of its initial activity after storage of the medium for at least about 4 months at temperatures from −20° C. to +50° C.,

4. The method of claim 1, wherein applying a medium to the wound involves applying a medium to damaged tissue resulting from a cause selected from the group consisting of thermal energy, electrical energy, chemical exposure, radiant energy or radiation.

5. The method of claim 1, wherein applying a medium to the wound involves applying a medium to damaged tissue having a cause selected from the group consisting of a chronic wound, a pressure sore, gangrene, elevated MMP concentrations, a diabetic ulcer, pressure ulcer, a varicose ulcer, infection, a postoperative wound, a burn, a carbuncle, and a pilonidal cyst wound.

6. The method of claim 1, wherein applying a medium to a wound, involves applying a medium further including an antimicrobial.

7. The method of claim 6, wherein applying a medium including the immobilized enzyme and an antimicrobial to the wound involves applying a medium including an antimicrobial selected from the group consisting of an antibiotic, an antifungal, an antiviral and an antiseptic.

8. The method of claim 1, wherein applying a medium to a wound, involves applying a medium further including a topical analgesic or anesthetic.

9. The method of claim 8, wherein applying a medium to a wound involves applying a medium further including a topical analgesic or anesthetic selected from the group consisting of acetaminophen, ibuprofen, lidocaine, bupivacaine, prilocaine, and aloe vera.

10. The method of claim 9, wherein applying a medium to a wound involves applying a medium further including an anti-inflammatory agent.

11. The method of claim 10, wherein applying a medium including an immobilized enzyme and an anti-inflammatory to a wound further involves applying the medium further including an anti-inflammatory selected from the group consisting of an NSAID, omega-3 fatty acid, an extract of white willow bark, curcumin, ginger, an extract of green tea, pycnogenol, Boswellia serrata resin, resveratrol, Uncaria tomentosa, and capsaicin.

12. The method of claim 1, wherein applying a medium including an immobilized enzyme to the wound involves treating the wound to provide healing.

13. A composition for the debridement of damaged tissue from a wound, disease, or other condition, including a medium, and an immobilized protease enzyme, wherein the medium is a fluid medium,
wherein the immobilized enzyme is a protease enzyme,
wherein the immobilized enzyme substantially resists degradation or deactivation in the wound, and
wherein the immobilized enzyme is stable to temperatures of at least 25° C.

14. The immobilized protease enzyme of claim 13 wherein the immobilized protease enzyme maintains at least 50% of its initial activity after storage for at least 4 months at temperatures from −20° C. to +50° C.

15. The composition of claim 13, wherein the composition further includes an enzyme that is not immobilized.

16. The composition of claim 13, wherein the composition includes a further immobilized enzyme.

17. The composition of claim 16, wherein the composition including a further immobilized enzyme includes a further immobilized enzyme located on a support containing the protease enzyme.

18. The composition of claim 16, wherein the composition including a further immobilized enzyme includes a further immobilized enzyme located on a support not containing the protease enzyme.

19. The composition of claim 13, wherein the medium is selected from the group consisting of a lotion, a salve, a cream, a slurry, a paste, and an aerosol.

20. The composition of claim 19, wherein the medium is contained on a material selected from the group consisting of a pad, a wraparound bandage, and a compress.

21. The composition of claim 13, wherein the medium further includes an antimicrobial selected from the group consisting of an antibiotic, an antifungal, an antiviral and an antiseptic.

22. The composition of claim 13, wherein the medium including the immobilized enzyme further includes a topical analgesic or anesthetic.

23. The composition of claim 13, wherein the medium including the immobilized further includes a topical analgesic or anesthetic selected from the group consisting of acetaminophen, ibuprofen, lidocaine, bupivacaine, prilocaine, and aloe vera.

24. The composition of claim 13, wherein the medium including the immobilized enzyme further includes an anti-inflammatory agent.

25. The composition of claim 13, wherein the medium including the immobilized enzyme further includes an anti-inflammatory selected from the group consisting of NSAID's, omega-3 fatty acid, extract of white willow bark, curcumin, ginger, extract of green tea, pycnogenol, Boswellia serrata resin, resveratrol, Uncaria tomentosa, and capsaicin.

26. A composition for the debridement of damaged tissue from a wound, condition, or disease, the composition including a fluid medium and an immobilized protease enzyme,
wherein the immobilized protease enzyme is derived from a protease enzyme, a crosslinker, and at least one polyfunctional material,
wherein the fluid medium is selected from the group consisting of a lotion, a salve, a paste, a cream, a slurry, and an aerosol,
wherein the immobilized enzyme debrides damaged tissue contacted.
